(19) United States
(12) Patent Application Publication
(10) Pub. No.: US 2002/0052039 A1
(43) Pub. Date: May 2, 2002

(54) Process for preparing an enzyme extract

(75) Inventors: Bettina Kirschbaum, Frankfurt (DE); Gotz Wilbert, Gersthofen (DE); Franz Effenberger, Stuttgart (DE)

Correspondence Address: Clariant Corporation, Industrial Property Department, 4331 Chesapeake Drive, Charlotte, NC, 28216, US

(73) Assignee: Clariant GmbH

(21) Appl. No.: 10/002,091

(22) Filed: Nov. 1, 2001

(30) Foreign Application Priority Data
Nov. 1, 2000 (DE) 10054012.0

(51) Int. Cl. C12N 9/78; C12N 9/88

(52) U.S. Cl. 435/232

(57) ABSTRACT

The present invention relates to a process for preparing an (R)-hydroxynitrile lyase extract by extracting an (R)-hydroxynitrile lyase-containing natural product with water in the absence or presence of a buffer at a pH of 3.3 to 5.5.

### BACKGROUND OF THE INVENTION

The invention relates to a process for preparing an (R)-hydroxynitrile lyase extract.

(R)-Hydroxynitrile lyase catalyzes the enantioselective addition of prussic acid to aldehydes, with the preparation of optically active cyanohydrins. Optically active cyanohydrins serve as building blocks for producing biologically active substances which are used, for example, in the pharmaceutical industry or agricultural industry (EP 0547655, EP 0276375).

(R)-Hydroxynitrile lyase may be produced from various natural sources. Enzyme and Microb. Technol. 1999, 25, 384-391 describes, in addition to the use of defatted almond flour, which is readily accessible in large amounts, the use also, inter alia, of apple cores and other less readily available fruit cores or stones.

Frequently, the extracted enzyme is purified before use, as specified in EP-0 547 655, or used fixed on a polymer support, as described in EP-0 276 375. Both enzyme preparation methods are relatively complex and thus have little attraction for industrial use.

The natural enzyme sources may also sometimes be used in situ, see Synth. Commun. 1991, 21, 1387-1391. Here, when used industrially, there is the problem that relatively large amounts of HCN-containing solids must be handled and disposed of.

The use of enzyme extract which is crude and therefore readily prepared, is described in Tetrahedron Lett. 1988, 29, 4485-4488. The aldehydes used as substrate generally comprise less sterically demanding aliphatic aldehyde, furfural and benzaldehyde and their 3- or 4-substituted derivatives. However, exceptions are 4-hydroxybenzaldehyde and 3,4-dihydroxybenzaldehyde which cannot be reacted, or can only be reacted to a small extent, to give the corresponding cyanohydrins, Top. Curr. Chem. 1999, 200, 193-226, in particular Table 2 on page 206.

2-substituted benzaldehydes are also not used in the enzyme-catalyzed, enantioselective cyanohydrin reaction with (R)-hydroxynitrile lyases, since under the customary reaction conditions only poor conversion rates and low ee values are obtained.

### SUMMARY OF THE INVENTION

In view of the above-described disadvantages, there is a need for a process for preparing an (R)-hydroxynitrile lyase extract which has a better activity than the (R)-hydroxynitrile lyase extracts prepared according to the prior art. The process must be simple to carry out and not make great demands on conversion to an industrial scale.

The object is surprisingly achieved by a process for preparing an (R)-hydroxynitrile lyase extract by extracting an (R)-hydroxynitrile lyase-containing natural product with water in the absence or presence of a buffer at a pH of 3.3 to 5.5.

The inventive process is a method which is simple and implementable industrially without special requirements for preparing an (R)-hydroxynitrile lyase extract whose activity permits aldehydes that can be converted into optically active cyanohydrins using known enzyme preparations to be reacted comparably or better. In addition, surprisingly, optically active cyanohydrins can be prepared with good conversion rates and high ee values from substrates which are difficult to react, such as 2-substituted benzaldehydes and 4-hydroxy and 3,4-dihydroxybenzaldehyde. In this case, unexpectedly high space-time yields are obtained for enzyme reactions.

The (R)-hydroxynitrile lyase-containing natural product used very successfully is comminuted fruit cores, in particular comminuted defatted fruit cores. Fruit cores are taken to mean fruit cores and fruit stones.

### DETAILED DESCRIPTION OF THE INVENTION

According to a particularly suitable variant, the (R)-hydroxynitrile lyase-containing natural product used is comminuted defatted apple cores or almond stones. However, other (R)-hydroxynitrile lyase-containing fruit cores or fruit stones, which are preferably comminuted and defatted, may also be used in the inventive process.

The inventive process uses, according to a preferred variant, defatted almond flour, as produced as waste product in the production of almond oil.

Enzyme extraction is carried out at a temperature of 0 to 60° C., in particular 10 to 50° C., preferably 20 to 40° C.

As mentioned above, the (R)-hydroxynitrile lyase-containing natural product is extracted with water in the absence or presence of a buffer. If no buffer is employed, care must be taken to ensure that the preset pH of 3.3 to 5.5 is maintained during the entire extraction. This is achieved, since the pH increases to values >5.5 during the extraction, by controlled addition of acid, for example a mineral acid, during the extraction. The water used for the extraction is also in advance set to an appropriate pH by addition of acid. A procedure of this type is given in example 5. pHs above 5.5 lead to extracts having reduced activity in the enantioselective cyanohydrin reaction, see example 4a (comparison example).

Usually, the (R)-hydroxynitrile lyase-containing natural product and water or aqueous buffer solution are used in a weight ratio of 1:(1 to 50), in particular 1:(2 to 20), preferably 1:(2.5 to 10).

In a number of cases it can be advantageous to extract the (R)-hydroxynitrile lyase-containing natural product in the presence of a buffer. Those which are particularly suitable are buffers or buffer mixtures which develop their buffer action in the specified pH range of 3.3 to 5.5 and keep the pH in this range during the extraction. If the buffering action of the buffer is insufficient to keep the pH in the preset range during the extraction, the pH must also be adjusted by acid addition.

Suitable buffers are, for example, glutamic acid-glutamate buffer, phosphoric acid-phosphate buffer, acetic acid-acetate buffer and citric acid-citrate buffer, in particular acetic acid-acetate buffer and citric acid-citrate buffer.

It has proved to be useful to carry out the extraction in the presence of 20 to 500 mmol of buffer/liter, in particular 40 to 300 mmol of buffer/liter, preferably 80 to 160 mmol of buffer/liter. Usually the buffer is dissolved in water and it is used in the form of an aqueous solution containing 20 to 500, in particular 40 to 300, preferably 80 to 160, mmol of buffer per liter.

The extraction is particularly simple when the pH of the buffer and the amount of the buffer are chosen so as to ensure that the predetermined pH range is maintained during the entire extraction.

The natural product can also be used in non-defatted form. In this case its proportion must be increased in relation to the buffer solution according to the fat content.

After an appropriate time of action of the water or the buffer solution on the natural product of, for example, 0.5 to 24, in particular 2 to 20, preferably 3 to 18, hours, the enzyme extract is usually separated from the natural product by filtration using a suitable filter apparatus.

The aqueous enzyme extract can be used together with the extracted natural product for the enantioselective HCN addition. However, it is expedient to separate off the extracted natural product and to utilize the aqueous enzyme extract freed from the natural product. In this manner natural product contaminated by HCN is avoided as waste.

Advantageously, a reaction may be carried out using an enzyme extract corresponding to the invention and a substrate, for example an aromatic aldehyde, in a two-phase system, with the substrate being used in an organic water-immiscible solvent in the presence of HCN.

When the reaction is carried out using the inventive enzyme extract, sufficient mixing must be ensured.

The aqueous enzyme extract can be separated off from the organic cyanohydrin-containing phase after the reaction is completed, and used in a following reaction.

The invention is described in more detail by the examples below, without being thereby restricted.

The activities of the enzyme extracts prepared were determined using a method of M. Bauer, H. Griengl and W. Steiner Biotechnol. Bioeng. 1999, 62, 23.

The ee values of the resulting cyanohydrins were determined after derivitization with acetic anhydride/pyridine by gas chromatography via a β-cyclodextrin column. (For the definition of 1 unit, see also K. Drauz, H. Waldmann Enzyme Catalysis in Organic Synthesis, Vol. 1, Verlag Chemie, Weinheim, 1995, p. 22.)

### EXAMPLES

Experimental Part

Enzyme is always taken to mean below (R)-hydroxynitrile lyase.

### Example 1

Preparation of Enzyme Extract Using 80 mmol Citrate Buffer/liter, pH 4.8

8.4 g of citric acid monohydrate are made up with demineralized water to 500 ml. The pH is adjusted to 4.8 with a few drops of 50% NaOH solution.

100 g of defatted almond flour are admixed with 500 ml of this citrate buffer and stirred for 16 hours at room temperature. The suspension is filtered through a glass frit. About 400 ml of aqueous enzyme extract are obtained having a pH of 5.2 and an activity of about 200 U/ml.

Preparation of (R)-2-chlorobenzaldehyde Cyanohydrin

56.2 g of 2-chlorobenzaldehyde (0.4 mol) are dissolved in 200 ml of diisopropyl ether and 200 ml of the enzyme extract (40,000 U) prepared above and 16.2 g of HCN (0.6 mol) are added. The reaction mixture is stirred vigorously at room temperature for 45 min, with an emulsion being formed. After stirring has ended, about 70% of the aqueous enzyme extract originally used is separated off. It is possible to use this extract for further reactions. The organic phase comprises (R)-2-chlorobenzaldehyde cyanohydrin (conversion rate according to GC 99%), having an ee of 83%.

### Example 2

Preparation of Enzyme Extract Using 80 mmol of Citrate Buffer/liter, pH 3.3

8.4 g of citric acid monohydrate are made up to 500 ml with demineralized water. The pH is adjusted to 3.3 using a few drops of 50% NaOH solution.

100 g of defatted almond flour are admixed with 500 ml of this citrate buffer and stirred at room temperature for 16 hours. The suspension is filtered through a glass frit. About 400 ml of aqueous enzyme extract are obtained, having a pH of 4.4 and an activity of about 75 U/ml.

Preparation of (R)-2-chlorobenzaldehyde Cyanohydrin

28.1 g of 2-chlorobenzaldehyde (0.2 mol) are dissolved in 100 ml of diisopropyl ether and 270 ml of the enzyme extract (20,000 U) prepared above and 8.1 g of HCN (0.3 mol) are added. The reaction mixture is stirred vigorously at room temperature for 90 minutes, with an emulsion being formed. After stirring has ended, about 70% of the aqueous enzyme extract originally used are separated off. It is possible to use this extract for further reactions. The organic phase contains (R)-2-chlorobenzaldehyde cyanohydrin (conversion rate according to GC 95%), having an ee of 82%.

### Example 3

Preparation of Enzyme Extract Using 160 mmol Citrate Buffer/liter, pH 4.8

16.8 g of citric acid monohydrate are made up to 500 ml with demineralized water. The pH is adjusted to 4.8 using a few drops of 50% NaOH solution.

100 g of defatted almond flour are admixed with 500 ml of this citrate buffer and stirred for 16 hours at room temperature. The suspension is filtered through a glass frit. About 400 ml of aqueous enzyme extract are obtained having a pH of 5.0 and an activity of about 200 U/ml.

Preparation of (R)-2-chlorobenzaldehyde Cyanohydrin

28.1 g of 2-chlorobenzaldehyde (0.2 mol) are dissolved in 100 ml of diisopropyl ether and 100 ml of the enzyme extract (20,000 U) prepared above and 8.1 g of HCN (0.3 mol) are added. The reaction mixture is stirred vigorously at room temperature for 45 minutes, with an emulsion being formed. After stirring has ended, about 70% of the aqueous enzyme extract originally used is separated off. It is possible to use this extract for further reactions. The organic phase contains (R)-2-chlorobenz-aldehyde cyanohydrin (conversion rate according to GC 98%), having an ee of 83%.

### Example 4

Preparation of Enzyme Extract Using 20 mmol Citrate Buffer/liter, pH 3.3

2.1 g of citric acid monohydrate are made up to 500 ml with demineralized water. The pH is adjusted to 3.3 using a few drops of 50% NaOH solution.

100 g of defatted almond flour are admixed with 500 ml of this citrate buffer and stirred at room temperature for 16 hours. The suspension is filtered through a glass frit. About 400 ml of aqueous enzyme extract having a pH of 5.0 and an activity of about 70 U/ml are obtained.

Preparation of (R)-2-chlorobenzaldehyde Cyanohydrin

28.1 g of 2-chlorobenzaldehyde (0.2 mol) are dissolved in 100 ml of diisopropyl ether and 285 ml of the enzyme extract (20,000 U) prepared above and 8.1 g of HCN (0.3 mol) are added. The reaction mixture is stirred vigorously at room temperature for 60 minutes, with an emulsion being formed. After stirring is ended, about 70% of the aqueous enzyme extract originally used is separated off. It is possible to use this extract for further reactions. The organic phase contains (R)-2-chlorobenzaldehyde cyanohydrin (conversion rate according to GC 98%), having an ee of 83%.

### Example 4a (Comparison Example)

Preparation of enzyme extract using 20 mmol citrate buffer/liter, pH 5.5 (based on the method described in Synth. Commun. 1991, 21, page 1388, but by means of extraction and removal of almond flour)

2.1 g of citric acid monohydrate are made up to 500 ml with demineralized water. The pH is set to 5.5 using a few drops of 50% NaOH solution.

100 g of defatted almond flour are admixed with 500 ml of this citrate buffer and stirred at room temperature for 16 hours. The pH increases markedly during the extraction, as demonstrated below in the case of the resultant enzyme extract. The suspension is filtered through a glass frit. About 400 ml of aqueous enzyme extract having a pH of 6.0 and an activity of about 70 U/ml are obtained.

Preparation of (R)-2-chlorobenzaldehyde Cyanohydrin

28.1 g of 2-chlorobenzaldehyde (0.2 mol) are dissolved in 100 ml of diisopropyl ether and 285 ml of the enzyme extract (20,000 U) prepared above and 8.1 g of HCN (0.3 mol) are added. The reaction mixture is stirred vigorously at room temperature for 60 minutes, with an emulsion being formed. After stirring is ended, about 70% of the aqueous enzyme extract originally used is separated off. The organic phase contains (R))-2-chlorobenzaldehyde cyanohydrin (conversion rate according to GC 99%), having an ee of 55%.

The activity of the enzyme extract is, as is shown clearly by a simple comparison of the ee values (ee=enantiomeric excess) in the preparation of (R)-2-chlorobenz-aldehyde cyanohydrin in example 4, is considerably lower and when an enzyme extract produced according to the invention is used.

### Example 5

Preparation of Enzyme Extract Using Aqueous Solution, pH 4.5-5.2 (without Buffer)

500 ml of demineralized water are adjusted to pH 4.5 using a few ml of concentrated HCl. 100 g of defatted almond flour are admixed with this solution and stirred at room temperature for 16 hours, with the pH being maintained in a range of 4.5-5.2 by continuous addition of concentrated HCl. The suspension is filtered through a glass frit. About 400 ml of aqueous enzyme extract having a pH of 4.8 and an activity of about 200 U/ml are obtained.

Preparation of (R)-2-chlorobenzaldehyde Cyanohydrin

28.1 g of 2-chlorobenzaldehyde (0.2 mol) are dissolved in 100 ml of diisopropyl ether and 100 ml of the enzyme extract (20,000 U) prepared above and 8.1 g of HCN (0.3 mol) are added. The reaction mixture is stirred vigorously at room temperature for 60 minutes, with an emulsion being formed. After stirring is ended, about 70% of the aqueous enzyme extract originally used is separated off. It is possible to use this extract for further reactions. The organic phase contains (R)-2-chlorobenzaldehyde cyanohydrin (conversion rate according to GC 99%), having an ee of 83%.

### Example 6

Preparation of Enzyme Extract Using 80 mmol Citrate Buffer/liter, pH 4.8

The enzyme extract is prepared as described in example 1.

Preparation of (R)-benzaldehyde Cyanohydrin

74.3 g of benzaldehyde (0.7 mol) are dissolved in 250 ml of diisopropyl ether and 100 ml of the enzyme extract (20,000 U) mentioned above and 27 g of HCN (1.0 mol). The reaction mixture is stirred vigorously at room temperature for 180 minutes, with an emulsion being formed. After stirring is ended, about 70% of the aqueous enzyme extract originally used is separated off. It is possible to use this extract for further reactions. The organic phase contains (R)-benzaldehyde cyanohydrin (conversion rate according to GC quantitative), having an ee of 98%.

### Example 7

Preparation of Enzyme Extract Using 80 mmol Citrate Buffer/liter, pH 4.8

The enzyme extract is prepared as described in example 1.

Preparation of (R)-3-hydroxybenzaldehyde Cyanohydrin

12.2 g of 3-hydroxybenzaldehyde (0.1 mol) are dissolved in 100 ml of diisopropyl ether and admixed with 100 ml of the enzyme extract (20,000 U) mentioned above and 4 g of HCN (0.15 mol). The reaction mixture is stirred vigorously at room temperature for 105 minutes, with an emulsion being formed. After stirring is ended, about 70% of the aqueous enzyme extract originally used is separated off. It is possible to use this extract for further reactions. The organic phase contains (R)-3-hydroxybenzaldehyde cyanohydrin (conversion rate according to GC 96%), having an ee of 97%.

### Example 8

Preparation of Enzyme Extract Using 80 mmol Citrate Buffer/liter, pH 4.8

The enzyme extract is prepared as described in example 1.

Preparation of (R)-3-hydroxybenzaldehyde Cyanohydrin

48.8 g of 3-hydroxybenzaldehyde (0.4 mol) are dissolved in 200 ml of diisopropyl ether and admixed with 100 ml of the enzyme extract (20,000 U) mentioned above and 16 g of HCN (0.6 mol). The reaction mixture is stirred vigorously at room temperature for 225 minutes, with an emulsion being formed. After stirring is ended, about 70% of the aqueous enzyme extract originally used is separated off. It is possible to use this extract for further reactions. The organic phase contains (R)-3-hydroxybenzaldehyde cyanohydrin (conversion rate according to GC 94%), having an ee of 92%.

### Example 9

Preparation of Enzyme Extract Using 80 mmol Citrate Buffer/liter, pH 4.8

The enzyme extract is prepared as described in example 1.

Preparation of (R)-4-hydroxybenzaldehyde Cyanohydrin

12.2 g of 4-hydroxybenzaldehyde (0.1 mol) are dissolved in 100 ml of diisopropyl ether and admixed with 100 ml of the enzyme extract (20,000 U) mentioned above and 4 g of HCN (0.15 mol). The reaction mixture is stirred vigorously at room temperature for 165 minutes, with an emulsion being formed. After stirring is ended, about 70% of the aqueous enzyme extract originally used is separated off. It is possible to use this extract for further reactions. The organic phase contains (R)-4-hydroxybenzaldehyde cyanohydrin (conversion rate according to GC 70%), having an ee of 92%.

### Example 9a (Comparison Example)

Preparation of Enzyme Extract Using 80 mmol Citrate Buffer/liter, pH 5.3

8.4 g of citric acid monohydrate are made up to 500 ml with demineralized water. The pH is set to 5.3 using a few drops of 50% NaOH solution.

100 g of defatted almond flour are admixed with 500 ml of citrate buffer and stirred at room temperature for 16 hours. The suspension is filtered through a glass frit. About 400 ml of aqueous enzyme extract having a pH of 5.7 and an activity of about 200 U/ml are obtained.

Preparation of (R)-4-hydroxybenzaldehyde Cyanohydrin

12.2 g of 4-hydroxybenzaldehyde (0.1 mol) are dissolved in 100 ml of diisopropyl ether and 100 ml of the enzyme extract (20,000 U) prepared above and 4 g of HCN (0.15 mol) are added. The reaction mixture is stirred vigorously at room temperature for 165 minutes, with an emulsion being formed. After stirring is ended, about 70% of the aqueous enzyme extract originally used is separated off. The organic phase contains (R)-4-hydroxybenzaldehyde cyanohydrin (conversion rate according to GC 63%), having an ee of 70%.

### Example 10

Preparation of Enzyme Extract Using 80 mmol Citrate Buffer/liter, pH 4.8

The enzyme extract is prepared as described in example 1.

Preparation of (R)-3,4-dihydroxybenzaldehyde cyanohydrin

6.9 g of 3,4-dihydroxybenzaldehyde (0.05 mol) are dissolved in 100 ml of diisopropyl ether and 100 ml of the enzyme extract (20,000 U) mentioned above and 3 g of HCN (0.1 mol) are added. The reaction mixture is stirred vigorously at room temperature for 165 minutes, with an emulsion being formed. After stirring is ended, about 70% of the aqueous enzyme extract originally used is separated off. It is possible to use this extract for further reactions. The organic phase contains (R)-3,4-dihydroxybenzaldehyde cyanohydrin (conversion rate according to GC 65%), having an ee of 76%.

1. A process for preparing an (R)-hydroxynitrile lyase extract by extracting an (R)-hydroxynitrile lyase-containing natural product with water in the absence or presence of a buffer at a pH of 3.3 to 5.5.

2. The process as claimed in claim 1, wherein the (R)-hydroxynitrile lyase-containing natural product is comminuted fruit cores.

3. The process as claimed in claim 1, wherein the (R)-hydroxynitrile lyase-containing natural product is comminuted defatted fruit cores.

4. The process as claimed in claim 1, wherein the (R)-hydroxynitrile lyase-containing natural product is comminuted defatted apple cores or almond stones.

5. The process as claimed in claim 1, wherein extraction is performed in the presence of 20 to 500 mmol buffer/liter.

6. The process as claimed in claim 1, wherein extraction is performed in the presence of 40 to 300 mmol buffer/liter.

7. The process as claimed in claim 1, wherein extraction is performed in the presence of 80 to 160 mmol buffer/liter.

8. The process as claimed in claim 1, wherein extraction is performed in the presence of a citric acid-citrate buffer.

9. The process as claimed in claim 1, wherein extraction is performed at a pH of 4.0 to 5.5.

10. The process as claimed in claim 1, wherein extraction is performed at a pH of 4.5 to 5.3.
